(19) United States
(12) Patent Application Publication
(10) Pub. No.: US 2022/0182394 A1
(43) Pub. Date: Jun. 9, 2022

(54) SYSTEM AND DEVICE FOR ENSURING THE AUTHENTICATION AND INTEGRITY OF GENOMIC DATA BASED ON BLOCK-CHAIN TECHNOLOGY

(71) Applicant: MyGenomeBox Co., Ltd, Incheon (KR)

(72) Inventors: Shang Cheol SHIN, Gwacheon-si (KR); Suk Chul BACK, Seoul (KR); In Cheol HAM, Incheon (KR)

(21) Appl. No.: 17/544,458

(22) Filed: Dec. 7, 2021

(30) Foreign Application Priority Data
Dec. 7, 2020 (KR) 10-2020-0169254
Aug. 18, 2021 (KR) 10-2021-0108669

(51) Int. Cl. H04L 9/08; H04L 9/32

(52) U.S. Cl. CPC H04L 63/123; H04L 9/0866; H04L 2209/38; H04L 9/3247; H04L 63/10

(57) ABSTRACT

A system and device use block-chain technology for ensuring the authentication and integrity of genomic data. The system includes a certification authority (CA) device, an analysis institution device, a user device, a utilization institution device, and a plurality of node devices. The CA device generates a pair of private and public keys to the analysis institution device and the user device. The analysis institution device performs genome analysis to extract genomic data, performs electronic signature for the genomic data with the analysis institution private key, and transmits the genomic data to the user device. The user device authenticates with the analysis institution public key, performs electronic signature with the user private key, and stores the genomic data. The utilization institution device transmits, to the user device, a smart contract that specifies a utilization institution identifier, and a utilization scope and period of the genomic data.

### CROSS-REFERENCE TO RELATED APPLICATION(S)

This application is based on and claims priority under 35 U.S.C. 119 to Korean Patent Application Nos. 10-2020-0169254, filed on Dec. 7, 2020, and 10-2021-0108669, filed on Aug. 18, 2021, in the Korean Intellectual Property Office, the disclosures of which are herein incorporated by reference in their entireties.

### BACKGROUND

### Technical Field

The present disclosure relates to a system and device for ensuring the authentication and integrity of genomic data based on block-chain technology.

### Description of Related Technology

Recently, with the advent of cryptocurrency, block-chain technology which is the basis of cryptocurrency is being actively studied. In addition, service conversion using block-chain technology is rapidly occurring in various fields of society. This is because anyone can easily use reliable services by breaking away from the existing centralized trust structure.

Currently, rather than developing a new system based on block-chain, block-chain technology is contributing to help a system meet a demand for changing the existing closed trust network to an open trust network or wishing to have a distributed DB.

### SUMMARY

The present disclosure is intended to provide a system and device using block-chain technology for ensuring the authentication and integrity of genomic data.

According to an embodiment of the disclosure, a system for ensuring authentication and integrity of genomic data based on block-chain technology may include a certification authority (CA) device, an analysis institution device, a user device, a utilization institution device, and a plurality of node devices. The CA device provides a module capable of generating a key pair of an analysis institution private key and an analysis institution public key to the analysis institution device and provides a module capable of generating a key pair of a user private key and a user public key to the user device. The analysis institution device performs genome analysis, extracts genomic data as a result of the genome analysis, performs electronic signature for the genomic data with the analysis institution private key, and transmits the genomic data to the user device. The user device authenticates with the analysis institution public key that the genomic data has been analyzed by the analysis institution device, performs electronic signature with the user private key, and stores the genomic data. The utilization institution device transmits, to the user device, a smart contract that specifies a utilization institution identifier, a utilization scope of the genomic data, and a utilization period of the genomic data. The plurality of node devices forms a network to configure a block-chain. In particular, the user device generates a transaction and transmits the transaction to the network of the node devices, the transaction including the smart contract, access right information for the genomic data, and a signature file in which the access right information is digitally signed with the user private key. In addition, one of the node devices that succeeds in generating a block stores the transaction in the generated block and propagates the block with the transaction to the node devices in the network. Also, the plurality of node devices in the network verify the block and, if verification is successful, connect the block to the block-chain.

In the system, the utilization institution device may receive the transaction from the network, confirm the transaction, and if there is an access right to a genomic data storage linked to the block-chain within the utilization scope and period of the genomic data, access the genomic data storage to download the genomic data.

According to an embodiment of the disclosure, a user device includes a processor. The processor may be configured to receive, from an analysis institution device, user's genomic data extracted as a result of a genome analysis, to receive, from a utilization institution device, a smart contract that specifies a utilization institution identifier, a utilization scope of the genomic data, and a utilization period of the genomic data, and to generate a transaction, the transaction including the smart contract, access right information for the genomic data, and a signature file in which the access right information is digitally signed with a user private key. In addition, the processor may be configured to transmit the transaction to a block-chain network formed of a plurality of node devices, whereby one of the node devices that succeeds in generating a block stores the transaction in the generated block and propagates the block with the transaction to the node devices in the block-chain network, and the plurality of node devices in the block-chain network verify the block and, if verification is successful, connect the block to the block-chain.

In the user device, the processor may be further configured to receive, from the analysis institution device, a serial number for which the analysis institution device performs electronic signature with an analysis institution private key, to add electronic signature to the received serial number with the user private key, to transmit, to the analysis institution device, the serial number to which electronic signature is added with the user private key; and to receive, from the analysis institution device, a genetic test kit having the serial number to which electronic signature is added with the user private key.

According to an embodiment of the disclosure, a utilization institution device includes a processor configured to transmit, to a user device, a smart contract that specifies a utilization institution identifier, a utilization scope of genomic data, and a utilization period of the genomic data, in case that the user device receives, from an analysis institution device, user's genomic data extracted as a result of a genome analysis. The processor may be further configured to receive a transaction from a block-chain network formed of a plurality of node devices, wherein the transaction is generated by the user device and includes the smart contract, access right information for the genomic data, and a signature file in which the access right information is digitally signed with a user private key, and wherein the user device transmits the transaction to the block-chain network, whereby one of the node devices that succeeds in generating a block stores the transaction in the generated block and propagates the block with the transaction to the node devices in the block-chain network, and the plurality of node devices in the block-chain network verify the block and, if verification is successful, connect the block to the block-chain. The processor may be further configured to download the genomic data by accessing a genomic data storage linked to the block-chain network.

In the utilization institution device, the processor may be further configured to upon receiving the transaction from the block-chain network, confirm the transaction, and if there is an access right to the genomic data storage linked to the block-chain network within the utilization scope and period of the genomic data, access the genomic data storage to download the genomic data.

According to the present disclosure, the user and the analysis institution can record the genome analysis request and the entire genome analysis process. Also, the user and the analysis institution can be clearly recorded through the electronic signature on the genomic data, which is the result of the genome analysis. Therefore, when using this data later, authentication can be made clear. In addition, according to the present disclosure, it is possible to restrict access to genomic data storage through block-chain, and to guarantee integrity by detecting contamination or falsification of data through both the analysis institution signature and the user signature on the genomic data.

### BRIEF DESCRIPTION OF THE DRAWINGS

FIG. 1 is a schematic diagram illustrating a system for ensuring the authentication and integrity of genomic data based on block-chain technology according to an embodiment of the present disclosure.

FIGS. 2, 3, and 4 are flow diagrams illustrating operations of devices for ensuring the authentication and integrity of genomic data based on block-chain technology according to an embodiment of the present disclosure.

### DETAILED DESCRIPTION

Electronic signature is based on an electronic handwritten signature and digital signature based on a discrete logarithmic problem and an elliptic curve algorithm. The electronic signature provides a non-repudiation function through the electronic handwritten signature and provides the integrity of an electronic document through a service such as time-stamping authority (TSA). However, there is no proper way to prove the electronic handwritten signature if a person denies it. In order to supplement this problem, a service that provides user identification and a non-repudiation function by combining an electronic document with a biometric-based electronic signature is being developed.

Hereinafter, embodiments of the present disclosure will be described in detail with reference to the accompanying drawings.

In the following description of embodiments, techniques that are well known in the art and not directly related to the present disclosure are not described. This is to clearly convey the subject matter of the present disclosure without obscuring it by omitting an unnecessary explanation.

In the drawings, the same or corresponding elements are denoted by the same reference numerals. In the following description of embodiments, elements can be expressed as a singular or plural form. Such singular or plural representations are selected appropriately according to situations presented for the convenience of description, and the present disclosure is not limited to the singular or plural form. Even expressed in a singular form, an element may be construed as a plurality of elements, and vice versa.

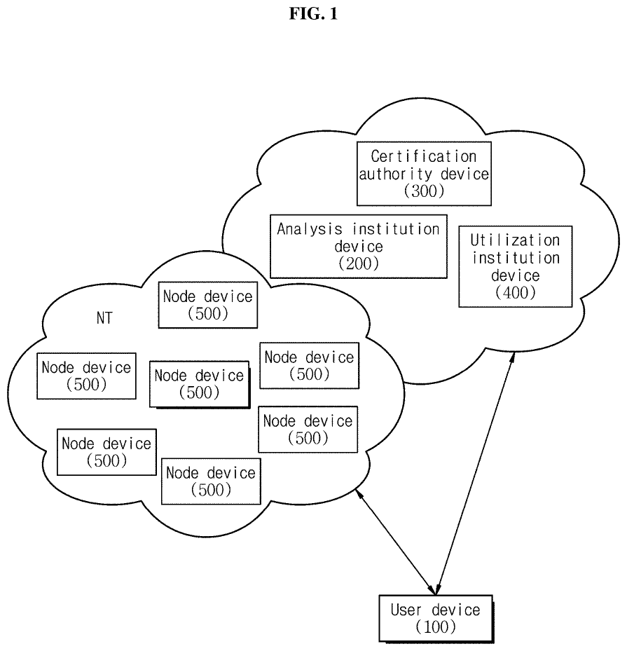

At the outset, a system for ensuring the authentication and integrity of genomic data based on block-chain technology according to an embodiment of the present disclosure will be described with reference to FIG. 1. FIG. 1 is a schematic diagram illustrating a system for ensuring the authentication and integrity of genomic data based on block-chain technology according to an embodiment of the present disclosure.

Referring to FIG. 1, the system includes a user device 100, an analysis institution device 200, a certification authority (CA) device 300, a utilization institution device 400, and a plurality of node devices 500.

The user device 100 refers to a device used by a user who requests genome analysis. The user device 100 generates a key pair of a user private key and a user public key by using a module provided by the CA device 300.

The analysis institution device 200 refers to a device used by an analysis institution that analyzes a user's genome and provides an analysis result. The analysis institution device 200 generates a key pair of an analysis institution private key and an analysis institution public key by using a module provided by the CA device 300. In addition, the analysis institution device 200 provides the user device 100 with genomic data which is a result of analyzing a user's genome.

The CA device 300 provides a module capable of generating a key pair of a user private key and a user public key to the user device 100, and provides a module capable of generating a key pair of an analysis institution private key and an analysis institution public key to the analysis institution device 200. In addition, the CA device 300 provides a module capable of generating a key pair of a utilization institution private key and a utilization institution public key to the utilization institution device 400.

The utilization institution device 400 refers to a device used by an institution that utilizes genomic data. For example, such utilization institutions may include, but are not limited to, a hospital, a university, a research institute, a healthcare service provider, and the like. The utilization institution device 400 generates a key pair of a utilization institution private key and a utilization institution public key by using a module provided by the CA device 300.

The plurality of node devices 500 are devices for forming a network NT (e.g., a peer-to-peer (P2P) network) to configure a block-chain.

Each of the user device 100, the analysis institution device 200, the CA device 300, the utilization institution device 400, and the node devices 500 is an apparatus capable of performing a computing operation and communicating with each other through a communication network. For example, each of the user device 100, the analysis institution device 200, the CA device 300, the utilization institution device 400, and the node devices 500 may be one of various computing devices such as a personal computer, a laptop computer, a handheld device, a mobile phone, a smart phone, a workstation, a server, and the like.

Each of the user device 100, the analysis institution device 200, the CA device 300, the utilization institution device 400, and the node devices 500 may be implemented in a distributed computing environment in which both local and remote computing systems perform tasks. The communication network may include various wired or wireless communication networks. Also, the communication network may include a mobile communication network and/or an Internet network.

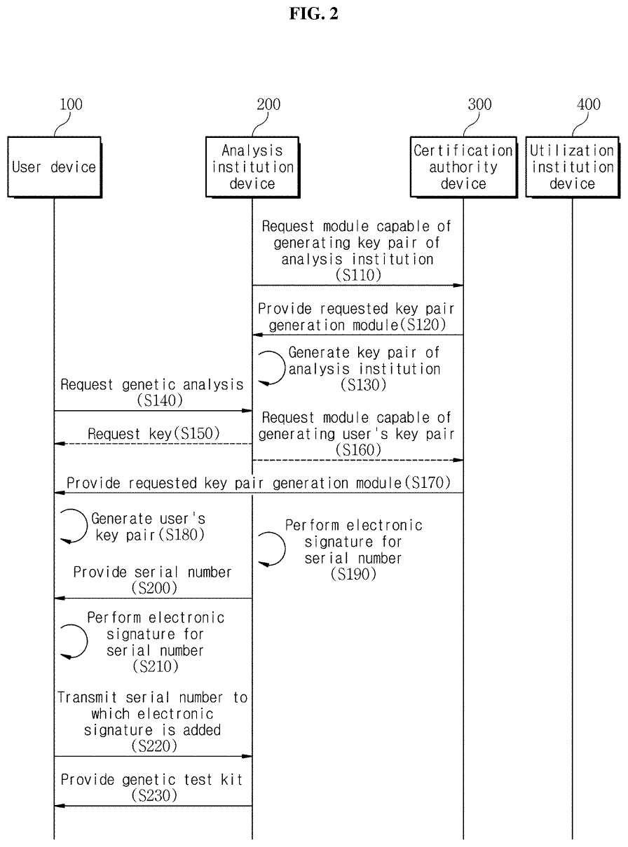

Next, operations of the above-described devices will be described in detail with reference to FIGS. 2 to 4. FIGS. 2, 3, and 4 are flow diagrams illustrating operations of devices for ensuring the authentication and integrity of genomic data based on block-chain technology according to an embodiment of the present disclosure.

First, as shown in FIG. 2, at step S110, the analysis institution device 200 requests a module capable of generating its own key pair from the CA device 300. This key pair is composed of an analysis institution public key and an analysis institution private key.

Then, at step S120, the CA device 300 provides the requested key pair generation module to the analysis institution device 200.

Next, at step S130, the analysis institution device 200 generates a key pair of the analysis institution private key and the analysis institution public key by using the module received from the CA device 300.

Meanwhile, at step S140, according to a user's manipulation, the user device 100 requests the analysis institution device 200 to perform an analysis of a user's genome.

Then, in order to induce the user to generate a user's key pair through the CA device 300, the analysis institution device 200 requests a key from the user device 100 at step S150 and also requests the CA device 300 to provide a module capable of generating a user key pair to the user device 100 at step S160.

Then, at step S170, the CA device 300 provides the requested key pair generation module to the user device 100.

Next, at step S180, the user device 100 generates a user key pair of a user private key and a user public key by using the module received from the CA device 300.

In addition, at step S190, the analysis institution device 200 assigns a serial number to the user and performs electronic signature for the serial number with the analysis institution private key. The serial number refers to a specific identification number given to an individual user by the analysis institution. The electronic signature refers to a process of encrypting a hash value with a private key and appending it. That is, at the step S190, the analysis institution device 200 encrypts a hash value of the serial number with its own private key and adds the encrypted hash value to the serial number.

Next, at step S200, the analysis institution device 200 provides the user device 100 with the serial number for which electronic signature is completed.

Upon receiving the serial number, the user device 100 confirms, by using the public key of the analysis institution device 200, that the electronic signature of the serial number is an electronic signature made by the analysis institution. In addition, at step S210, with the user private key, the user device 100 performs electronic signature for the serial number assigned to the user.

Next, at step S220, the user device 100 transmits the serial number, to which electronic signature is added with the user private key, to the analysis institution device 200.

Then, at step S230, the analysis institution device 200 issues a tag having the serial number received from the user device 100, and provides a genetic test kit attaching the tag to the user device 100.

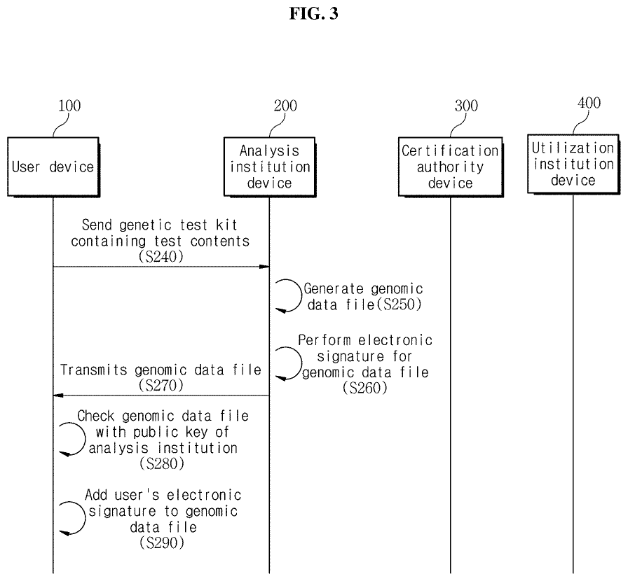

Subsequently, referring to FIG. 3, the user device 100 checks and authenticates whether the tag having the serial number to which electronic signature has been added with the user private key is attached to the genetic test kit received from the analysis institution device 200. Then, at step S240, the user device 100 sends the genetic test kit containing test contents (i.e., a material derived from the human body, such as saliva) to the analysis institution.

Next, at step S250, the analysis institution device 200 authenticates the tag of the genetic test kit received from the user and performs genome analysis with the test contents contained in the kit. At this time, the analysis institution device 200 generates a genomic data file by extracting genomic data according to the analysis result.

Then, at step S260, the analysis institution device 200 performs electronic signature for the genomic data file. Upon completing the electronic signature, at step S270, the analysis institution device 200 transmits the genomic data file to the user device 100.

Then, at step S280, the user device 100 checks the received genomic data file with the public key of the analysis institution. If there is no abnormality, the user device 100 adds user's electronic signature to the genomic data file at step S290. That is, the user device 100 authenticates with the analysis institution public key that the genomic data has been analyzed by the analysis institution, performs electronic signature with the user's own private key, and stores the genomic data file.

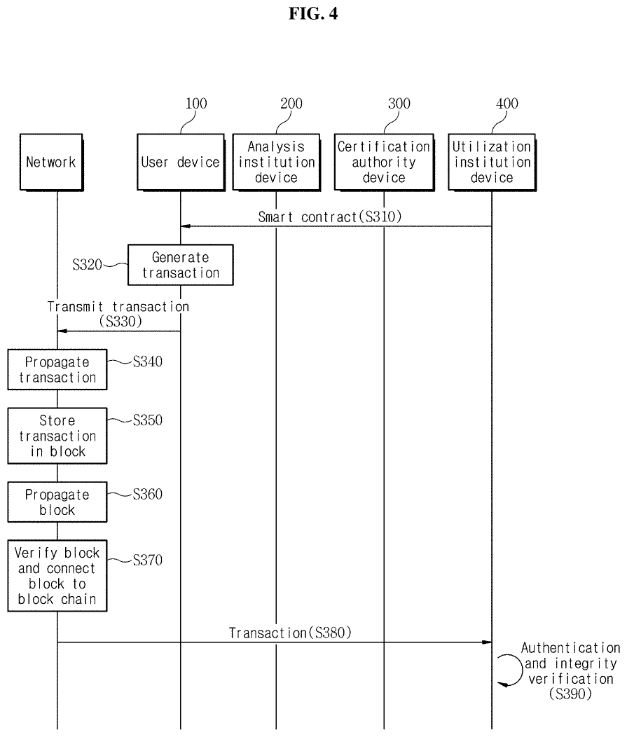

Meanwhile, referring to FIG. 4, at step S310, the user device 100 may receive, from the utilization institution device 400, a smart contract for requesting consent to utilize the genomic data. The smart contract may be provided in the form of an electronic document file, for example. The smart contract specifies at least one of a utilization institution identifier (e.g., institution name, electronic signature, etc.), a utilization scope of genomic data, and a utilization period of genomic data.

Upon receiving the smart contract, the user device 100 generates a transaction based on the smart contract at step S320. Specifically, the user device 100 generates the transaction, which includes the smart contract, access right information for the genomic data, and a signature file in which the access right information is digitally signed with the user's private key.

Next, at step S330, the user device 100 transmits the transaction to the block-chain network NT, e.g., to one of the node devices 500 in the block-chain network NT. In this case, the user device 100 may insert or add the user public key in or to the transaction to be transmitted. Alternatively, the user public key may be shared in advance with the plurality of node devices 500 belonging to the block-chain network NT.

Then, at step S340, one of the node devices 500 receiving the transaction from the user device 100 propagates the transaction throughout the block-chain network NT.

When any one node device among the plurality of node devices 500 belonging to the block-chain network NT succeeds in block generation (i.e., mining), the node device 500 that succeeds in block generation stores the transaction in the generated block at step S350. At this time, the user identifier of the user who provides the genomic data, the utilization institution identifier of the utilization institution that utilizes the genomic data, the utilization scope of the genomic data, the utilization period of the genomic data, etc. may be recorded in the block.

In addition, the node device 500 that succeeds in block generation and stores the transaction in the block propagates the block to the block-chain network NT at step S360.

Then, at step S370, the plurality of node devices 500 belonging to the block-chain network NT verify the block and, if the verification is successful, connect the block to the existing block-chain.

Meanwhile, the utilization institution device 400 may receive the transaction from the block-chain network NT at step S380. If there is an access right to a genomic data storage (linked to the block-chain) within the utilization scope and period of the genomic data by confirming the transaction, the utilization institution device 400 can access the genomic data storage and download the genomic data.

After downloading the genomic data, the utilization institution device 400 performs authentication and integrity verification at step S390 based on the public keys issued by the user and the analysis institution. That is, the utilization institution device 400 can check the electronic signature of the genomic data and thereby detect the authenticity of the user and the analysis institution and whether the genomic data is contaminated or tampered with.

In the above, the operations of several devices for ensuring the authentication and integrity of genomic data based on block-chain technology according to an embodiment of the present disclosure have been described. It will be understood that each block of the flowchart illustrations, and combinations of blocks in the flowchart illustrations, may be implemented by computer program instructions. These computer program instructions may be provided to a processor of a general purpose computer, special purpose computer, or other programmable data processing apparatus to produce a machine, such that the instructions, which are executed via the processor of the computer or other programmable data processing apparatus, generate means for implementing the functions specified in the flowchart block(s). These computer program instructions may also be stored in a computer usable or computer-readable memory that may direct a computer or other programmable data processing apparatus to function in a particular manner, such that the instructions stored in the computer usable or computer-readable memory produce an article of manufacture including instruction means that implement the function specified in the flowchart block(s). The computer program instructions may also be loaded onto a computer or other programmable data processing apparatus to cause a series of operational steps to be performed on the computer or other programmable apparatus to produce a computer implemented process such that the instructions that are executed on the computer or other programmable apparatus provide steps for implementing the functions specified in the flowchart block(s). In addition, each block of the flowchart illustrations may represent a module, segment, or portion of code, which comprises one or more executable instructions for implementing the specified logical function(s). It should also be noted that in some alternative implementations, the functions noted in the blocks may occur out of the order. For example, two blocks shown in succession may in fact be executed substantially concurrently or the blocks may sometimes be executed in the reverse order, depending upon the functionality involved.

While the disclosure has been particularly shown and described with reference to exemplary embodiments thereof, it will be understood by those skilled in the art that various changes in form and details may be made therein without departing from the scope of the subject matter as defined by the appended claims.

What is claimed is:

1. A system for ensuring authentication and integrity of genomic data based on block-chain technology, the system comprising:
a certification authority (CA) device providing a module configured to generate a key pair of an analysis institution private key and an analysis institution public key to an analysis institution device and providing a module configured to generate a key pair of a user private key and a user public key to a user device;
the analysis institution device configured to perform genome analysis, extract genomic data based on the genome analysis, perform electronic signature for the genomic data with the analysis institution private key, and transmit the genomic data to the user device;
the user device configured to authenticate with the analysis institution public key that the genomic data has been analyzed by the analysis institution device, perform electronic signature with the user private key, and store the genomic data;
a utilization institution device configured to transmit, to the user device, a smart contract that specifies a utilization institution identifier, a utilization scope of the genomic data, and a utilization period of the genomic data; and
a plurality of node devices forming a network to configure a block-chain,
wherein the user device is configured to generate a transaction and transmit the transaction to the network of the node devices, the transaction including the smart contract, access right information for the genomic data, and a signature file in which the access right information is digitally signed with the user private key,
wherein one of the node devices that succeeds in generating a block is configured to store the transaction in the generated block and propagate the block with the transaction to the node devices in the network, and
wherein the plurality of node devices in the network are configured to verify the block and, in response to verification being successful, connect the block to the block-chain.

2. The system of claim 1, wherein the utilization institution device is configured to receive the transaction from the network, confirm the transaction, and in response to an existence of an access right to a genomic data storage linked to the block-chain within the utilization scope and period of the genomic data, access the genomic data storage to download the genomic data.

3. A user device comprising:
a processor configured to:
receive, from an analysis institution device, user's genomic data extracted based on a genome analysis;
receive, from a utilization institution device, a smart contract that specifies a utilization institution identifier, a utilization scope of the genomic data, and a utilization period of the genomic data;
generate a transaction, the transaction including the smart contract, access right information for the genomic data, and a signature file in which the access right information is digitally signed with a user private key; and
transmit the transaction to a block-chain network comprising a plurality of node devices, such that one of the node devices that succeeds in generating a block stores the transaction in the generated block and propagates the block with the transaction to the node devices in the block-chain network, and the plurality of node devices in the block-chain network verify the block and, in response to verification being successful, connect the block to the block-chain.

4. The user device of claim 3, wherein the processor is further configured to:
receive, from the analysis institution device, a serial number for which the analysis institution device performs electronic signature with an analysis institution private key;
add electronic signature to the received serial number with the user private key;
transmit, to the analysis institution device, the serial number to which electronic signature is added with the user private key; and
receive, from the analysis institution device, a genetic test kit having the serial number to which electronic signature is added with the user private key.

5. A utilization institution device comprising:
a processor configured to:
transmit, to a user device, a smart contract that specifies a utilization institution identifier, a utilization scope of genomic data, and a utilization period of the genomic data, in response to the user device receiving, from an analysis institution device, user's genomic data extracted based on a genome analysis;
receive a transaction from a block-chain network comprising a plurality of node devices, wherein the transaction is generated by the user device and includes the smart contract, access right information for the genomic data, and a signature file in which the access right information is digitally signed with a user private key, and wherein the user device transmits the transaction to the block-chain network, such that one of the node devices that succeeds in generating a block stores the transaction in the generated block and propagates the block with the transaction to the node devices in the block-chain network, and the plurality of node devices in the block-chain network verify the block and, in response to verification being successful, connect the block to the block-chain; and
download the genomic data by accessing a genomic data storage linked to the block-chain network.

6. The utilization institution device of claim 5, wherein the processor is further configured to:
upon receiving the transaction from the block-chain network, confirm the transaction, and in response to an existence of an access right to the genomic data storage linked to the block-chain network within the utilization scope and period of the genomic data, access the genomic data storage to download the genomic data.
